(19) United States
(12) Patent Application Publication
(10) Pub. No.: US 2014/0298495 A1
(43) Pub. Date: Oct. 2, 2014

(54) METHOD FOR OBTAINING A TRIPLOID INTERSPECIFIC HYBRID OF MEAGRE AND RED DRUM

(71) Applicant: Georges Balma, Sete (FR)

(72) Inventor: Georges Balma, Sete (FR)

(21) Appl. No.: 14/006,923

(22) PCT Filed: Mar. 19, 2012
(86) PCT No.: PCT/FR2012/050579

§ 371 Date: Oct. 7, 2013

(30) Foreign Application Priority Data
Mar. 23, 2011 (FR) 11 52396

(51) Int. Cl. A01K 67/027; A01K 67/02

(52) U.S. Cl. CPC A01K 67/027; A01K 67/02
USPC 800/8; 119/217

(57) ABSTRACT

The invention relates to a method for obtaining a triploid interspecific hybrid of meagre and red drum belonging to the family of the Sciaenidae. The method includes the following two steps: a) crossing a meagre Argyrosomus regius with a red drum Sciaenops ocellatus, and b) carrying out treatment to induce triploidy in the issue obtained.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### BACKGROUND OF THE INVENTION

1. Field of the Invention

The invention relates to a method for obtaining triploid interspecific hybrid fish from meagre fish and another fish pertaining to the Sciaenidae family.

The present invention is related to the field of aquaculture and, more particularly to fish farming.

2. Description of Related Art Including Information Disclosed Under 37 CFR 1.97 and 37 CFR 1.98

The quantitative contribution of world-wide fishing to the aquatic product markets, namely for covering the humans' fish consumption, has been stable for about the last twenty years. But especially the present state of the stocks of wild fishes does generally not permit, because of an unfortunately increasingly frequent overfishing, to expect a change in this tendency, in particular in a context of an increase of the world population.

Therefore, the future supply of the world market relies on the upswing of aquaculture and in particular on the upswing of fish farming, which designates more specifically the rearing of fishes. Though the reared aquatic products have nowadays increased significantly their market share over the last twenty years, the improvement of the species proposed for fish farming and their diversification remain a fundamental necessity.

In the Atlantic and the Mediterranean regions, the marine fish farming relates to a wide variety of different fish species, and namely Atlantic salmon (Salmo salar), sea-bass (Dicentrarchus labrax), sea-bream (Sparus aurata), but also Bluefin tuna (Thunnus thynnus), halibut (Hippoglossus hippoglossus), soles (Solea solea and Solea senegalensis), codfish (Gadus morua) or also meagre fish (Argyrosomus regius).

The meagre fish, or Argyrosomus regius, pertains to the Sciaenidae family. This is a European marine fish, which is rare in fishing. The yearly catches are varying and, though they can reach 10 000 tons, this remains a minor contribution to the volumes of European fishing. In addition, the size of the fishes caught is largely varying. These characteristics induce an increase in prices, which reflects the consumers' interest for the meagre fish.

The meagre fish possesses indeed a very much appreciated fine and flavorsome flesh, the quality of which is comparable to that of other reared marine species. The low rate of lipids and their balance in the muscle provides the species with a high dietetic value. This species thus possesses done undeniable gustatory and nutritional qualities.

Its gaining a position on the market is thus potentially restricted by its low availability. The more that the meagre fish has the characteristics of a good candidate for aquaculture, because, in addition to the quality of its flesh, it has a quick growth under European latitudes, leading to a commercial size within a reasonable period, even though the information on the biology of the species in captivity is limited, because of its low expansion. However, the aquaculture production of meagre fish is recent and limited to the French Mediterranean coast, Corsica and Italy. This production only reached 1 000 tons a year in 2004.

Finally, the morphology of meagre fish makes that the treatment of its carcass, with a view to its transformation for example into deep-frozen products ready for consumption, is subject to much waste.

The inventors have managed to control the reproduction in captivity of meagre fish. However, solutions remained to be found to improve the species in order to maximize its aquaculture potential.

In the field of fish farming, a known technique for obtaining large-size animals is the blocking of the sexual maturation. This technique permits, on the one hand, to avoid the food ingested by the fishes from being diverted to the reproduction function; it is then used exclusively for the growing of the animal. On the other hand, the sexual maturation is responsible for physiological changes in the animal, which will induce a diminution in quality of the product.

As regards the aquaculture of codfish (Gadus morua), the blocking of the maturation occurs by maintaining the animals under artificial light. However, this technique has proved inconclusive.

Another known technique consists in carrying out an interspecific hybridization. This technique has advantages over the intraspecific hybridization, which can result into problems of loss of genetic diversity. Interspecific hybridization is used for the production of sterile animals, which avoids an uncontrolled reproduction between the domestic strains and the wild stocks. This permits to protect the natural environments against an eventual gene or environmental pollution related to the rearing of domestic strains or non-native strains. In addition, interspecific hybridization does not lead to genetic manipulation of the product. Indeed, the marketing of a genetically modified organism would require obtaining a previous administrative authorization and could disturb the consumer.

In the state of the technique is known, for example from WO 2006/067356, a method consisting in hybridizing a Dicentrarchus labrax sea-bar with a Dicentrarchus punctatus sea-bar, then in carrying out a triploidy-inducing treatment on the progeny being obtained.

There are also methods for triploidizing a sea-bar (Dicentrarchus labrax), which permit to obtain about 100% triploid individuals, namely through hybridization between tetraploid and diploid individuals. These techniques permit to improve some quality characteristics, such as the dressing percentage. However, the growth performances of the sea-bar triploids are generally 20% lower than those of the diploid references.

In addition, no method presently permits to improve the qualities of the meagre fish.

### SUMMARY OF THE INVENTION

The invention provides the possibility of providing a solution for the need to improve the intrinsic qualities of the meagre fish with a view to its aquaculture production, while coping with the various drawbacks of the state of the technique.

To this end, the invention relates to a method for obtaining a triploid interspecific hybrid fish from a meagre fish and a drum fish pertaining to the Sciaenidae family, comprising the following two steps:

a) hybridizing an Argyrosomus regius meagre fish with a Sciaenops ocellatus drum fish

b) carrying out a triploidy-inducing treatment on the progeny being obtained.

This method permits to obtain sterile hybrids. Furthermore, it is not an essentially biological method. Indeed, the triploidy-inducing treatment is an essential technical step of the method, which requires a human intervention, and is transposable at industrial scale.

Advantageously, the triploidy-inducing treatment consists in subjecting the fertilized eggs to a hyperbaric shock or a thermal shock.

Preferably, the hyperbaric shock is carried out at a pressure between 400 and 800 bars for 1 to 4 minutes, and the thermal shock is carried out at a temperature between −5° C. and +5° C. for 10 to 30 minutes.

According to an advantageous feature, the triploidy-inducing treatment is carried out between 2 and 10 minutes after fertilizing the eggs.

Advantageously, the method according to the invention relates to hybridizing a male Argyrosomus regius meagre fish with a female Sciaenops ocellatus drum fish or a female Argyrosomus regius meagre fish with a with a male Sciaenops ocellatus drum fish.

The invention also relates to a triploid hybrid from meagre fish and drum fish obtained by means of the method according to the invention. The advantages deriving from the present invention reside, essentially, in obtaining a sterile hybrid having a good yield in rearing and advantageous morphological characteristics. In addition, the profile of the hybrid regarding its adaption to more varied temperatures than the species from which it results is quite interesting, and provides a real added value for the conditions of rearing in Europe.

Further aims and advantages of the present invention will become clear from the following description, which will be complemented by the following figures.

### BRIEF DESCRIPTION OF THE DRAWINGS

FIG. 1 is a graph illustration showing the diet of hybrids in the larval phase during the first 30 days of their life, according to the modalities of example 2 hereafter.

FIG. 2 is another graph illustration showing the rearing temperature applied to the hybrid larvae of example 2, during the first 29 days of their life.

FIG. 3 is still another graph illustration showing the rearing temperature applied to the hybrid larvae of example 2, from day 30 to day 50 of the rearing.

FIG. 4 is a graph illustration showing the temperature of the basins during the nursery and growth period of example 3.

FIG. 5 is a graph illustration showing the growth curve from day 50 to day 114 for the groups of hybrid fry of example 3, with the reference meagre fish.

FIG. 6 is a photographic illustration, showing hybrids obtained by means of the method being described, on day 29 of their life at the left and on day 64 of their life at the right.

FIG. 7 is another photograph illustration, showing a hybrid obtained by means of the method being described, on day 114 of their life.

### DETAILED DESCRIPTION OF THE DRAWINGS

The present invention is related to the field of aquaculture, and more particularly to fish farming. The invention relates in particular to obtaining a triploid interspecific hybrid fish from a meagre fish and another fish pertaining to the Sciaenidae family, the tropical red drum (Sciaenops ocellatus).

The Sciaenidae family, to which the meagre fish and the drum fish, in particular the tropical red drum, pertain, is a large family of fishes grouping more than 200 species distributed into about 70 types.

The Argyrosomus type comprises namely the Argyrosomus regius species, usually referred to as “meagre fish”. The meagre fish is a marine fish from the Atlantic Ocean and the Mediterranean Sea. It has a fusiform body, slightly flattened on the flanks. Its back profile is clearly convex, and its ventral line is practically rectilinear. The color of the back is grey, the flanks and the dorsal fin are silver grey and shiny. A side line has a dotted black strip that extends until the caudal fin. The eyes are small, the snout has a large mouth. It possesses highly visible canines. In a characteristic way, the dorsal fin of the meagre fish is in two parts, the rear dorsal fin is clearly longer than its front dorsal fin. Its most common average size is between 50 centimeters and 1 meter, but it can reach 2 meters for 100 kilograms in a natural environment. It is caught in the coastal zone at less than 80 meters, on sandy or muddy bottoms. Since the meagre fish has a preference for the water layer closest to the bottom, its way of life is considered as semi-pelagic with a demersal tendency. A gregarious behavior is observed during the juvenile phase as well as during the migrations for reproduction. Outside this period, the adults seem to live isolated or in small groups. The sexual maturation is reached between 3 and 5 years (at 4-5 kg). The reproduction takes place from October to May at latest. The meagre fish does not resist temperatures lower than 10° C. In aquaculture, the meagre is reared between about 19° C. and 20° C.

The Sciaenops type comprises namely the Sciaenops ocellatus species, commonly referred to as “drum fish” and more particularly “tropical drum fish”, or also “red drum”. The tropical drum fish mainly lives in the coastal and estuarine waters from the north-east of the Atlantic Ocean to the Gulf of Mexico. Since its habitat in natural environment is completely different from that of the meagre fish, it is not likely for both species to meet. The tropical drum fish has a characteristic orange-red color, which may vary from grey to bronze red and has the peculiarity of having one or several black spots taken from the basis of the caudal peduncle, referred to as ocellus. The tropical drum fish can reach a size up to more than 1 meter, even 1.50 meters and can weigh about 40 kg. The tropical drum fish is an eurytherm and euryhaline, carnivorous species. The larvae essentially eat invertebrates, while the adults prefer fishes, shrimps and crabs. The juveniles are found in the bays and the estuaries until their sexual maturity, the fishes living further north move to the south in winter, in order to find warmer waters. The sexual maturation is reached between 3 and 5 years (at 4-5 kg). The reproduction takes places from August to October, i.e. during a period completely offset with respect to the period of reproduction of the meagre fish. The tropical drum fish does not withstand temperatures below 5° C. In aquaculture, the tropical drum fish is reared between 27° C. and 28° C. The drum fish thus possesses a thermal profile wider than that of the meagre fish, since, though it is adapted to warm water, it withstands colder conditions better than the meagre fish.

The hybridization between two members of one and the same phylogenetic family, in particular when these two members have periods of reproduction that do not overlap, requires the implementation of the following general steps:

collecting the sperm of one of the two species and preserving it in a cool environment or deep-frozen,

controlling the state of maturation of the gonads of the females of the other species,

collecting the eggs of the females, for example through abdominal pressure,

if necessary, stabilizing and controlling the hygrometry and the temperature of the oocytes being recovered,

defrosting the sperm of the male species (if deep-frozen) and controlling its quality and its mobility,

artificially fertilizing the oocytes with the sperm. Incubating the eggs being obtained, while regularly controlling the incubation and the cell division,

controlling the hatching,

larval rearing,

weaning,

nursery, ending into the growth phase.

The hybrid being obtained is neither like the meagre fish nor like the drum fish. Rather, said hybrid possesses some morphological characteristics of the meagre fish and others of the drum fish. The triploid hybrid has namely a body shape closer to that of the meagre fish and a color that is more similar to that of the drum fish.

The following examples show a detailed embodiment of the invention.

### Example 1

### Carrying Out of an Hybridization Between an Argyrosomus regius and an Sciaenops ocellatus

The reproduction of the two species does not take place at the same time, the hybridization occurs through artificial reproduction with deep-freezing of the sperm of the males of one species, waiting for the females of the second species to enter into reproduction. The hybridization can take place in both directions, the males of one species being hybridized with the females of the second species, and vice-versa.

In a traditional way, the female drum fishes have been induced through hormone injection. To this end, the females exhibiting an adequate egg maturation phase are selected, and are injected with the LHRHa (luteinostimulin-releasing hormone) at a dose of 10 mg/kg live weight, then they are isolated in an egg-deposition structure. Seventy hours after the induction of the females, the male and female gametes are recovered, and artificial fertilizing is then carried out by first mixing the oocytes and the sperm before adding sea water in the respective proportions of 1000:1:500.

At that time is carried out the induction of the triploidy of the eggs through hyperbaric or thermal shock.

The hyperbaric shock is preferably carried out at a pressure between 400 and 800 bars for 1 to 4 minutes.

Alternatively, the fertilized eggs can be subjected to a thermal shock, at a temperature between −5° C. and 5° C. for 10 to 30 minutes.

Generally, the treatment aiming the induction of the triploidy is carried out between 2 and 10 minutes after the fertilization of the eggs. The hybrids resulting from this method are triploid hybrids at about 100%.

Nevertheless, in order to check the triploidy and/or to evaluate the triploidy-induction performances, one can proceed to analyzing the cells of the hybrid to be tested, either in flow cytometry with propidium iodide or through visual analysis of their karyotype. The checking through flow cytometry can be performed with a Facscan-type (Becton Dickinson) flow cytometer. The DNA contents of each fish is then measured and compared with the cells of the reference sample (2n diploid), proceeding for example from a reference stock not subjected to the above-described triploidy-induction treatment. If one proceeds through karyotyping, it is enough to count the number of chromosomes the tested hybrid possesses, the triploids having 3n chromosomes, i.e. 72 chromosomes, the diploids having only 2n, i.e. 48 chromosomes. These measuring techniques by flow cytometry and/or karyotyping are conventional ones for the specialist in the art.

The triploid hybrids being obtained have a general morphology close to that of the meagre fish associated with a scale color including yellow reflections characteristic of the drum fish.

The specialist in the art can thus identify and select, by means of a simple visual control, the hybrids being obtained and distinguish the triploid hybrids from the hybrids that could not have been made triploid.

The specialist in the art can also contemplate the use of molecular markers present at the level of the DNA to identify the hybrids obtained by the method according to the invention.

The induced fertilized eggs are then incubated. After hatching, one proceeds to the larval rearing.

### Example 2

### Larval Rearing of the Hybrid Larvae Obtained Through Hybridizing Female Drum Fishes with Male Meagre Fishes

The larvae used proceed from eggs fertilized in Martinique. The larvae have been received in four 30-liter cubitainers at the age of 1 day, in Palavas, France, where the larval rearing was carried out. They were put into rearing in four cylindrical-conical 500-liter basins in closed circuit (the tank 1 contained reference drum fishes and the tanks 2, 3, 4 contained hybrids). They have not been counted, so as not to add a dangerous handling immediately after the important stress from the trip. The rearing conditions are detailed below.

Light Conditions

From day 1 (J1) to day 35 (J35), the animals are subjected to constant varying-intensity lighting.

From J1 to J10, the light intensity generated by the industrial white neon tubes is 300 lux at the surface of the water. In addition, a daily supply of algae limits the light penetration into the rearing environment, while stabilizing same (green water).

From J11 to J35, the light intensity is reduced to 50 lux by means of a shade cloth, while the algae are eliminated.

From day 35 (J35) to day 40 (J40), the larvae are progressively brought to an alternation 16 h day/8 h night in 3 successive steps (22 h day/2 h night; 19 h day/5 h night and finally 16 h day/8 h night).

Hydraulic Conditions

The renewal of the water is fixed at 3% at the beginning of the rearing, at 15% at 10 days and increases to 25% as from day 16. It is ensured through a water inlet in the subsurface oriented towards the wall, in order to avoid creating a current in the basin. The water outlet is at the surface, a slight air bubbling on the bottom of the basin ensures convection movement.

During all this period, surface skimmers are in place on the basins, in order to eliminate the oily layer generated by the food.

The salinity is natural (35-38 g/l) and physical-chemical analyses of the water are performed twice a week.

Feeding Conditions

The larvae are fed only with rotifers from their arrival until the age of 6 days. From day 7 to day 10, they are fed with a mixture of rotifers—Artemia nauplii (A1) and remain on A1 until the age of 15 days. From day 16, a small quantity of micro-particles (Gemma 0.1) is added to the food, then progressively increased until full weaning at the age of 31 days according to the sequence.

| Date | Food | Proportion (%) |
|---|---|---|
| J16-J18 | Gemma 0.1 | 100 |
| J19-J22 | Gemma 0.1/Gemma 0.2 | 60/40 |
| J23-J26 | Gemma 0.2 | 100 |
| J27-J31 | Gemma 0.2/Gemma 0.3 | 50/50 |

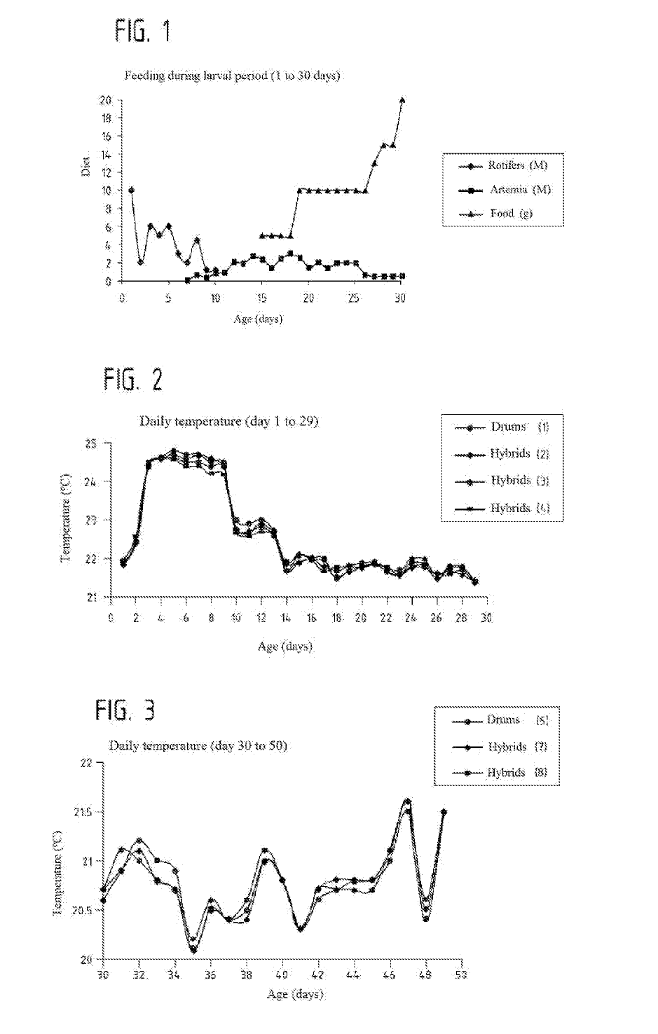

The food sequence is shown in FIG. 1, in which the unity of the “Rotifers” and “Artemia” curves is one million individual living prey a day and the inert food unity, represented by the “Food” curve, is the gram/day.

Temperature Conditions

After the trip at cool temperature (22° C.), the larvae quickly pass from 22° C. to 24° C. and are maintained at about 24° C. until the age of 9 days.

This temperature has been chosen between the rearing temperatures of the original species (the drum fish being reared between about 27 and 28° C. and the meagre fish between about 19 and 20° C.).

From day 10 to day 50, the temperature is adjusted between 21 and 22° C., in order to test the performances of the hybrid in metropolitan conditions. The graphic in FIG. 2 indicates the daily variations observed during the rearing on the four 500-liter basins during the first 29 days. At 30 days, further to a period of cannibalism, the hybrids have been sorted, counted and separated into two batches. The batch of the biggest one is then formed of 318 individuals and the batch of the smallest ones of 203 individuals. The basin of the drum fishes contains only 7 individuals. The daily measured temperatures between day 30 and day 50 of rearing in the 3 basins are represented in the graphic of FIG. 3.

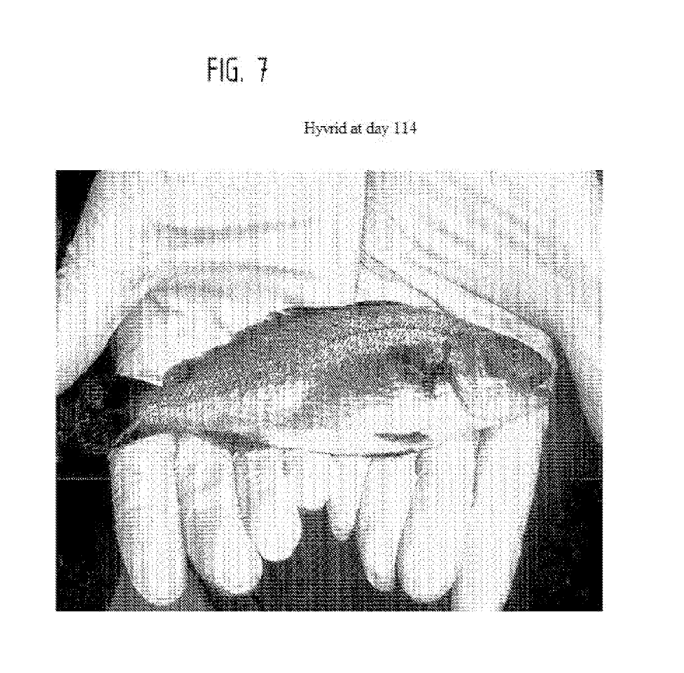

FIG. 6 on the left represents hybrids obtained by means of the described method, on day 29 of their life.

The hybrids have surprisingly withstood the rearing conditions. In particular, mortality has been reduced to only a few individuals over the duration of the weaning process (about ten out of a population evaluated at less than 1000 individuals).

### Example 3

### Nursery and Growth of the Fry Proceeding from the Larval Rearing of Example 2

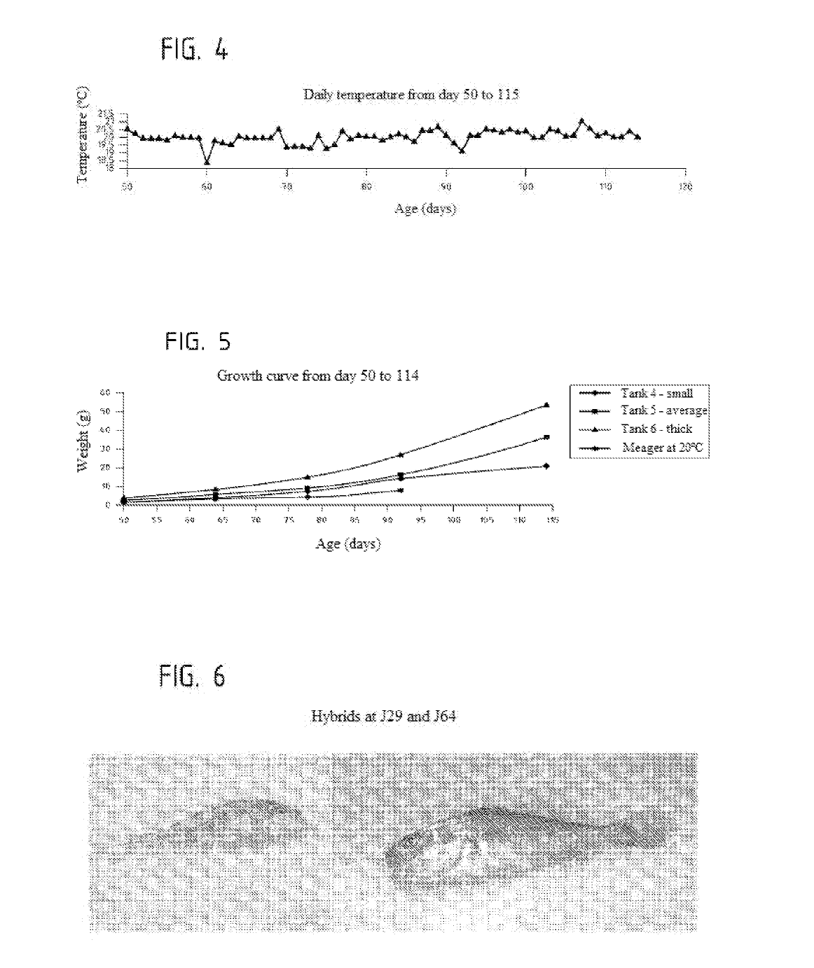

On day 50, the fry proceeding from the larval rearing described in example 2 is transferred into a nursery room provided with square 450-liter basins with a flat bottom, which operate in closed circuit. The light intensity is 50 lux and the temperature is as an average 20° C. with the variations shown in the graph of FIG. 4, which indicates the daily measured temperatures between day 50 and day 114 of the rearing.

Three batches of hybrids are formed:

batch of “small”: 112 individuals with an average weight of 1.52 g

batch of “average”: 199 individuals with an average weight of 2.49 g

batch of “thick”: 199 individuals with an average weight of 3.94 g.

The animals are fed in the beginning with a mixture of Gemma micro 0.3/A11 (Le Gouessant) by means of distributor belts, then quickly used to the self-feeders only on ‘le Gouessant’ food (A11; A12). On J50, the apparition of lesions on the caudal and dorsal fins can be due to food deficiencies, the animals have been fed with Skretting 1.9 food, which is generally used for the meagre fish. The rate of rationing is about 10% a day. It seems that the change of granulates had a favorable effect on the state of the fishes.

These rearing conditions have permitted to obtain the growth curves represented in FIG. 5. These curves have been established based on exhaustive biometrics or by sampling of 30 individuals at a bi-weekly rate.

FIG. 6 on the right represents hybrids obtained by means of the described method, on day 64 of their life.

It is observed that the hybrids—small, average or thick—advantageously have a more considerable growth than the meagre fish, in metropolitan conditions, at least until day 92.

From the age of 114 days, the fishes are placed in 5 m3-basins, in closed circuit and at a temperature of 20° C. The remaining stock is then 499 individuals.

The hybrids being obtained possess a fine flesh with high commercial potential, quick growth and offer the advantage of being able to adapt to more varied thermal profiles than the meagre fish. This is an undeniable advantage for the subsequent commercial development of the hybrids according to the invention, since the latter can be reared in highly diverse thermal conditions.

The morphology of the hybrid being obtained, which possesses a reduced head compared to the meagre fish, as can be seen in FIG. 7, permits to reach important gains on the transformation yields, which represents a very interesting advantage over the initial aim, which was obtaining a hybrid with a high commercial potential.

As is clear from the preceding description, the present invention solves the problem raised in a very relevant way. In particular, it permits the diversification of the stock available for fish farming, with a hybrid with a higher market value. It should be recalled that the aquatic products have an important role to play in the developing countries, in which the world consumption of animal proteins has been more than doubled over the last 30 years.

Indeed, the rearing of new “species” in fish farming is justified by an improvement in adapting to the varied conditions of environment observed in Europe and in the World, thanks to the availability of a wide range of species. Also thanks to these new “species”, an increase of the performances observed in rearing can be expected. Furthermore, the diversification of the stock permits to better meet the demands of the distributors and consumers, while permitting, as such is the case for the hybrid being obtained, which permits an easier and quicker transformation, a better productivity.

1. Method for obtaining a triploid interspecific hybrid fish from a meager fish and a drum fish pertaining to the Sciaenidae family, comprising the following two steps:
hybridizing an Argyrosomus regius meagre fish with a Sciaenops ocellatus drum fish; and
carrying out a triploidy-inducing treatment on the progeny being obtained from the step of hybridizing.

2. Method according to claim 1, wherein said Argyrosomus regius meagre fish is male and wherein said Sciaenops ocellatus drum fish is female.

3. Method according to claim 1, wherein said Argyrosomus regius meagre fish is female, and wherein said Sciaenops ocellatus drum fish is male.

4. Method according to claim 1, wherein said triploidy-inducing treatment is comprised of subjecting fertilized eggs to a hyperbaric shock.

5. Method according to claim 4, wherein the hyperbaric shock is carried out at a pressure between 400 and 800 bars for 1 to 4 minutes.

6. Method according to claim 1, wherein said triploidy-inducing treatment is comprised of subjecting fertilized eggs to a thermal shock.

7. Method according to claim 6, wherein the thermal shock is carried out at a temperature between −5° C. and +5° C. for 10 to 30 minutes.

8. Method according to claim 1, wherein said triploidy-inducing treatment is carried out between 2 and 10 minutes after fertilizing of eggs.

9. Triploid hybrid from meager fish and drum fish obtained according to the method of claim 1.
